(19) United States
(12) Patent Application Publication
(10) Pub. No.: US 2025/0352622 A1
(43) Pub. Date: Nov. 20, 2025

(54) GLP-1 FORMULATIONS AND THEIR USES

(71) Applicant: Rose Pharma Inc., Virginia Beach, VA (US)

(72) Inventors: Enda Kenny, Dublin (IE); Richard Warburg, St. Thomas, VI (US)

(73) Assignee: Rose Pharma Inc., Virginia Beach, VA (US)

(21) Appl. No.: 19/208,126

(22) Filed: May 14, 2025

Related U.S. Application Data
(60) Provisional application No. 63/648,006, filed on May 15, 2024.

(51) Int. Cl. A61K 38/26; A61P 3/04

(52) U.S. Cl. CPC A61K 38/26; A61P 3/04

(57) ABSTRACT

The GLP-1 agonist ROSE-010 may be administered in a manner that results in less side effects, and/or be more effective to achieve certain desired results than some other GLP-1 agonists, for example longer acting GLP-1 agonists.

### PRIORITY

The present application claims priority to U.S. provisional application No. 63/648,006 filed on May 15, 2024, which is incorporated herein by reference in its entirety.

### FIELD

The present disclosure relates to formulations of GLP-1 and GLP-1 agonists thereof and method of using such formulations.

### BACKGROUND

PCT Patent Application Publication No. WO/2011/017554 discloses “a pharmaceutical composition comprising GLP-1, a GLP-1 analog including the Val⁸ glucagon-like peptide-1 (7-37) OH (ROSE-010) having the following amino acid sequence: H-His-Val-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg-Gly-OH (ROSE-010; SEQ ID NO: 1) wherein Val (8) GLP-1 is a truncated version of human GLP-1.”

U.S. Pat. No. 8,642,548 states “[i]n one embodiment, the formulation comprising the active ingredient can be administered to the patient in a dry powder formulation by inhalation using a dry powder inhaler such as the inhaler disclosed, for example, in U.S. Pat. No. 7,305,986 and U.S. patent application Ser. No. 10/655,153 (US 2004/0182387), which disclosures are incorporated herein by reference. Repeat inhalation of dry powder formulation comprising the active ingredient can also be administered between meals and daily as needed. In some embodiments, the formulation can be administered once, twice, three or four times a day”, “[t]he method of treating hyperglycemia, diabetes, and/or obesity can be designed so that the patient can receive at least one dose of ROSE-10 formulation in proximity to a meal or snack” and “[i]n embodiments described herein, GLP-1 can be administered at mealtime (in proximity in time to a meal or snack). In this embodiment, GLP-1 exposure can be limited to the post-prandial period so it does not cause the long acting effects of the current therapies.”

### FIGURES

FIG. 1 illustrates a design of the study of Example 2.

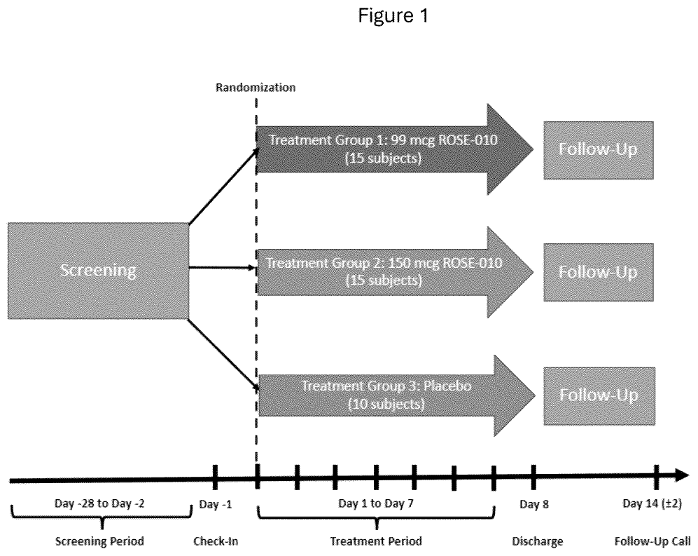

FIGS. 2A-B show that the ROSE-010 treated groups consistently eat less food weight (A) and less calories (B) than the placebo group. LD corresponds to 99 μg ROSE-010 treated group; HD corresponds to 150 μg ROSE-010 treated group; PL corresponds to the placebo group.

FIGS. 3A-B show plasma concentrations of ROSE-010 over time following sub-cutaneous injection: 100 ug (Cohort 1) and 150 ug (Cohort 2) ROSE-010.

FIGS. 4A-B show mean plasma concentrations versus time profiles (+standard deviation) for ROSE-010 on Day 7.

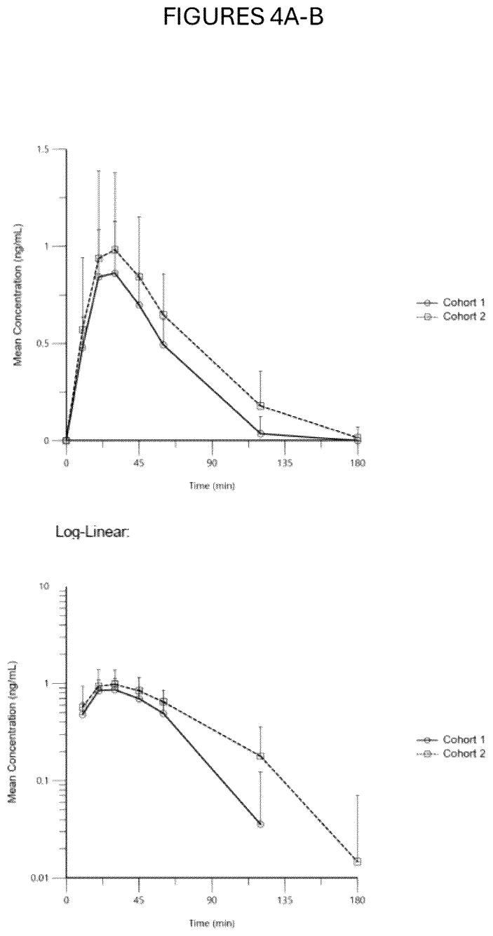

### SUMMARY

The present disclosure is based, at least in part, discoveries that ROSE-010 (and/or compounds similar to ROSE-010) can have advantageous properties and/or actions when it is administered under certain circumstances and/or conditions. For example, in certain aspects and embodiments of the disclosure, ROSE-010 may be administered in a manner that results in less side effects, and/or be more effective to achieve certain desired results than some other GLP-1 agonists, for example longer acting GLP-1 agonists. Surprisingly, Applicants have found that the side effects of ROSE-010 are significantly reduced when used to treat obese people while still suppressing appetite for extended periods, such as for 1, 2, 3 and 4 hours. As used herein, the term “ROSE-010” means a 31 amino acid peptide that includes the sequence of SEQ ID NO: 1. The Rose-010 sequence is identical to native glucagon-like-peptide-1 (GLP-1) (7-37) with position 8 substituted by a valine. ROSE-010 may in certain embodiments be administered in a formulation or administration route such as described, for example in WO/2011/017554 and/or 8,642,548, hereby incorporated by reference in their entirety. In certain embodiments, ROSE-010 is a short acting GLP-1 agonist as the term is defined herein.

As used herein, the term “short acting GLP-1 agonist” or “shorter-acting GLP-1 agonist” refers to a GLP agonist that, when administered to a human, is present and active in the human for no more or less than about 6 hours, preferably no more or less than 4 hours, no more or less than 3 hours or even no more or less than 1 or 2 hours. It has a plasma half-life ((T_{1/2}) of no more or less of than about 180 minutes or no more or less than about 120 minutes; or no more or less than about 90 minutes; or no more or less than about 75 minutes; or no more or less than about 60 minutes; or no more or less than about 45 minutes or about 30 minutes. It has a dosing frequency of once a week, once every other day or once a day, but can also be greater than once per day, such as twice a day. Shorter acting GLP-1 agonists include, but are not limited to ROSE-010 (SEQ ID NO: 1, a terminal half-life of about 30 minutes), exenatide (a half-life of about 2.5 hours) and lixisenatide (a half-life of about 3 hours). It is recognized that duration of activity can also be shortened by reducing the dosage amount or providing carriers that are immediate release, as opposed to selecting a GLP-agonist with a shorter circulatory half-life. These shorter-acting GLP-1 agonists, in many embodiments have a shorter duration of therapeutic plasma concentrations and in some embodiments may also result in a faster onset of action. In some embodiments, a short acting GLP-1 agonist may be suitable for daily administration; or more than once daily administration; or more than twice daily administration.

As used herein, the term “long acting GLP-1 agonist” or “longer-acting GLP-1 agonist” refers to a GLP-1 agonist when administered to a human, has a plasma half life ((T_{1/2}) of more than about 3 hours; or more than 3.5 hours or more than about 4 hours or more than about 5 hours; or more than about 6 hours or more than about 8 hours or more than about 10 hours or more about 12 hours. In some embodiments, longer acting GLP-1 agonists may include semaglutide (marketed as Ozempic® and Wegovy®), dulaglutide, tirzepatide (marketed as Zepbound® and Monjaro®), and liraglutide (marketed as Saxenda®). These longer-acting GLP-1 agonists have a greater duration of therapeutic plasma concentrations that results in less frequent dosing. In some embodiments, a long acting GLP-1 agonist as contemplated herein may be suitable for administration less frequently than once per day, for example once every two days; once every three days, or once weekly or even monthly.

Accordingly, in one aspect, a method of treating a subject is provided wherein the method includes administering to the subject a composition comprising ROSE-010 or an equivalent GLP-1 agonist prior to a meal.

As used herein, the term “ROSE-010 or an equivalent GLP-1 agonist” means ROSE-010 or a GLP-1 agonist that has structural and/or functional properties that are similar or comparable to ROSE-010. In certain embodiments an equivalent GLP-1 agonist is a short acting GLP agonist according to the disclosure.

In another aspect, a method is provided that includes identifying a subject that discontinued treatment with a longer acting GLP-1 agonist and administering to said subject a composition comprising ROSE-010 or an equivalent GLP-1 agonist. In one embodiment, a method is provided that includes identifying a subject that discontinued treatment with a longer acting GLP-1 agonist selected from the group consisting of semaglutide, dulaglutide, tirzepatide, and liraglutide and administering to said subject a composition comprising ROSE-010 or an equivalent GLP-1 agonist.

As used herein, such ROSE-010 was administered in an amount sufficient to reduce appetite of the subject and thereby help in maintenance of body weight or even reduction weight. By maintenance of weight is meant that the average weight of the patient from prior to administration of ROSE-010 is kept from being increased by more than 15%, preferably more than 10% or even 5% and most preferably within 0-4%. Such maintenance is preferably achieved with little loss of muscle mass and preferably gain in muscle mass. The amount administered may depend on the route of administration. Generally, the amount administered may be (plus or minus 10%) 100 micrograms, 125 micrograms, 150 micrograms, 175 micrograms, 200 micrograms, 225 micrograms, 250 micrograms or 300 micrograms when administered subcutaneously.

Another aspect of the disclosure provides a method comprising identifying a subject that had previously taken a longer acting GLP-1 agonist for at least 6 months, or at least 9 months, or at least a year and subsequently discontinued the GLP-1 longer acting agonist treatment, and administering to said subject a composition comprising ROSE-010 or an equivalent GLP-1 agonist. In one exemplary embodiment, a method is provided comprising identifying a subject that had previously taken a longer acting GLP-1 agonist selected from the group consisting of semaglutide, dulaglutide, tirzepatide, and liraglutide for at least 6 months, or at least 9 months, or at least a year and subsequently discontinued the GLP-1 longer acting agonist treatment, and administering to said subject a composition comprising ROSE-010 or an equivalent GLP-1 agonist.

In another aspect, provided is a method for reducing the amount of a long acting GLP-1 agonist selected from the group consisting of semaglutide, dulaglutide, tirzepatide, and liraglutide administered to a subject; said method comprising administering to said subject a composition comprising ROSE-010 or an equivalent GLP-1 agonist and reducing either or both of amount or frequency of the dose of said longer acting GLP-1 agonist administered to said subject. In some examples, the long acting GLP-1 is administered at half or one quarter the prior frequency (e.g., every other day instead of every day, or every third or fourth day), and the ROSE-010 is administered just prior to the main meal of that subject's day, or before two or even three of the meals that day. Such ROSE-010 can be administered on the days in which the long acting GLP-1 is not expected to be active such as a day or two days after its last administration.

Another aspect of the disclosure is a method for reducing at least one adverse side effect of a longer acting GLP-1 agonist administered to a subject; said method comprising administering to said subject a composition comprising ROSE-010 or an equivalent GLP-1 agonist and reducing either or both of amount or frequency of the dose of said long acting GLP-1 agonist administered to said subject to reduce the adverse side effects of said long acting GLP-1 agonist. In some embodiments, a method is provided for reducing at least one adverse side effect of a longer acting GLP-1 agonist selected from the group consisting of semaglutide, dulaglutide, tirzepatide, and liraglutide administered to a subject; said method comprising administering to said subject a composition comprising ROSE-010 or an equivalent GLP-1 agonist and reducing either or both of amount or frequency of the dose of said long acting GLP-1 agonist administered to said subject to reduce the adverse side effects or the time period of side effects being experienced compared to said long acting GLP-1 agonist. By adverse side effect is meant to include any or all of nausea, vomiting, gastrointestinal discomfort and a general feeling of sickness. ROSE-010 is able to generally exert its affect on appetite suppression with little or no such effects compared to those observed with longer acting GLP-1s. Moreover, such reduction is over a basis of 24 hours such that ROSE-010 may have any such side effect only for a short period of time such as for 1 or 2 hours and preferably less than 3 or 4 hours. For the remainder of the day the patient will not be subject to such side effects. In comparison longer acting GLP-1s can have an affect throughout the day or even over several days or even a month.

Another aspect of the disclosure involves a method for treating an overweight or obese subject, said method comprising administering a long acting GLP-1 agonist until the subject loses weight to a target level; then reducing or discontinuing dosing of said long acting GLP-1 agonist and administering to said subject a composition comprising ROSE-010 or an equivalent GLP-1 agonist. In some embodiments methods for treating an overweight or obese subject may involve administering a long acting GLP-1 agonist selected from the group consisting of semaglutide, dulaglutide, tirzepatide, and liraglutide until the subject loses weight to a target level; then reducing or discontinuing dosing of said long acting GLP-1 agonist and administering to said subject a composition comprising ROSE-010 or an equivalent GLP-1 agonist.

In another aspect of the disclosure is a method is provided that includes identifying a subject desiring to (or in need of) maintaining a particular weight and administering to said subject a composition comprising ROSE-010 or an equivalent GLP-1 agonist.

In another aspect, a method of maintaining weight in a subject is provided, said method comprising identifying a subject that had lost weight while taking a longer acting GLP-1 agonist selected from the group consisting of semaglutide, dulaglutide, tirzepatide, and liraglutide and administering to said subject a composition comprising ROSE-010 or an equivalent GLP-1 agonist.

A method is provided in yet another aspect that comprises identifying a patient diagnosed with, or at risk of, a cognitive disease or disorder and administering to said subject a composition comprising ROSE-010 or an equivalent agonist.

In a further aspect, a method is provided that includes identifying a patient diagnosed with, or at risk of, a neurodegenerative disease or disorder and administering to said subject a composition comprising ROSE-010 or an equivalent GLP-1 agonist.

Another aspect is a method of reducing a weight of an overweight or obese subject by administering to the subject a composition comprising ROSE-010 or an equivalent GLP-1 agonist prior to a meal in an amount sufficient to suppress the appetite of said subject.

Yet another aspect is a method of reducing food consumption in a subject desiring to reduce food consumption by administering to the subject a composition comprising ROSE-010 or an equivalent GLP-1 prior to a meal in an amount sufficient to suppress the appetite of said subject. In certain embodiments, the administering performed for 7 days once a day may result in at least 25% reduction of calorie consumption by the subject.

In some embodiments, a single event of said administering in the above methods does not result in a side effect associated with ROSE-010, such as vomiting and/or nausea, lasting longer than 3 hours or longer than 2.5 hours.

Further scope, applicability and advantages will become apparent from the non-restrictive detailed description given hereinafter. It should be understood, however, that this detailed description, while indicating exemplary embodiments or aspects, is given by way of example only.

### DETAILED DESCRIPTION

ROSE-010 is a 31 amino acid peptide (SEQ ID NO: 1) identical to native Glucagon-like-peptide-1 (GLP-1) (7-37) but position 8 has been substituted by valine. Under certain conditions and in some embodiments, the sequence of ROSE-010 renders the molecule resistant (or at least more resistant) to dipeptidyl peptidase IV (DPP IV) degradation. In certain formulations and conditions, this amino acid substitution may result in a plasma half-life (T1/2) of approximately 1 hour in the human following subcutaneous (s.c.) injection. ROSE-010 has been proposed for treatment of acute pain in IBS using an aqueous formulation or a dry powder.

ROSE-010 has been shown to exhibit no significant effect in female IBS-C patients on gastric volumes, small bowel or colonic transits at 24 h or bowel functions, but significantly retarded gastric emptying at 100 and 300 ug. Camilleri et al 303 American Journal of Physiology, Gastrointestinal and Liver Physiology G120-128, 2012.

Longer acting GLP-1 agonists, including semaglutide, dulaglutide, tirzepatide, and liraglutide are described in U.S. Pat. Nos. 7,762,994, 9,884,093, 9,474,780, and 8, 114,833, respectively.

Semaglutide has a t_{1/2} of about 1 week. Dulaglutide has a t_{1/2} of about 90 hours. Tirzepatide has a t_{1/2} of about 5 days. Liraglutide has a t_{1/2} of 12 hours.

The use of the terms “a” and “an” and “the” are to be construed to cover both the singular and the plural, unless otherwise indicated herein or clearly contradicted by context.

Unless specifically stated or obvious from context, as used herein the term “or” is understood to be inclusive and covers both “or” and “and”.

The term “and/or” where used herein is to be taken as specific disclosure of each of the specified features or components with or without the other.

The terms “comprising”, “having”, “including”, and “containing” are to be construed as open-ended terms (i.e., meaning “including, but not limited to”) unless otherwise noted. The term “consisting of” is to be construed as close-ended.

The term “about” or “approximately” shall generally mean within 20 percent, within 10 percent, within 5, 4, 3, 2 or 1 percent of a given value or range.

As used herein, the term “liquid” with respect to formulations means that the peptide is dissolved in a solution without significant precipitation.

As used herein, the term “stable” with respect to a formulation refers to a formulation in which no significant aggregation and/or degradation of the GLP-1 agonist occurs either in absence of stress or under one or more stress conditions.

Certain aspects and embodiments of the present disclosure are based, at least in part, on the finding that administration of shorter acting formulations of GLP-1 agonists is unexpectedly effective for reducing appetite and/or reducing weight, while reducing side effects associated with longer acting formulations of GLP-1 agonists.

As used herein, the term “GLP-J agonist” refers to a compound that activates a GLP-1 receptor and/or has structural similarity to GLP-1. In certain aspects and embodiments ROSE-010 is a GLP-1 agonist of the disclosure. The term further includes a GLP-1 agonist with a dual action such as a GIP/GLP-1 agonist.

Certain GLP-1 agonists can delay stomach emptying. However, for long acting GLP-1 agonists, after long term use the GLP-1 receptor may be always activated, and the effect of the GLP-1 agonist on the stomach emptying effect becomes reduced. Without being bound by any theory, it is believed that the primary effect on appetite is via the brain appetite centers. Short acting agonists, such as ROSE-010, on the other hand, will not always activate the receptors and will thus preserve the stomach emptying effect and so lead to a feeling of fullness in a different way, a way that is more similar to the way natural GLP-1 works in the body.

As used herein, the term “fast acting” refers to the ability of the GLP-1 agonist to exert its appetite suppression effect on a subject in a short amount of time, such as within 30 minutes. In a preferred embodiment, the short acting GLP-1 agonist is also fast acting, such as ROSE-010.

As used herein, the term “dosage period” refers to the period of time between dosage administration of a drug to a subject. For example, a long acting GLP-1 agonist may have a dosage period of one week, such that a subject is administered the agonist once a week. A short-acting GLP-1 agonist (such as ROSE-010), on the other hand, has shorter dosage periods such that it may be administered multiple times a day with significantly lower side effects, especially period of side effects.

### ROSE-010 GLP-1 Agonist & Formulations

The ROSE-010 agonist has the amino acid sequence (SEQ ID NO: 1). ROSE-010 is also fast acting and starts to exert its effects within 30 minutes with a terminal half-life of about 30 minutes. It acts by delaying stomach emptying, which predicts appetite reduction. Pharmaceutical formulations of the short acting GLP-1 agonist may be delivered in various manners, such as by injection, orally, nasally, or by inhalation. In accordance with some embodiments of the present disclosure, the compositions of the short acting GLP-1 agonist may be administered in the form of the stable liquid formulations as previously described in U.S. patent application Ser. No. 17/334,419, the disclosure of which is incorporated herein by reference in its entirety. In another embodiment, ROSE-010 or a short acting GLP-1 agonist may be administered in a dry powder formulation by inhalation using a dry powder inhaler such as the inhaler disclosed, for example, in WO 2011/017554, the disclosure of which is incorporated herein by reference in its entirety.

In one embodiment, the short acting GLP-1 agonist is present in a dosage that minimizes any adverse side effects. For example, the short acting GLP-1 agonist ROSE-010 may be present in the pharmaceutical composition in a dose of about 150 ug, about 100 ug, or about 75 to about 225 ug, or about 75 ug to 175 ug or about 100 ug to about 150 ug.

According to an embodiment, the short acting GLP-1 agonist may be administered multiple times a day, such as at least twice per day, or at least three times per day, or at least four times per day. Alternatively, the short acting GLP-1 agonist may be administered only once per day to cover a time period relating to a single meal. In some embodiments, the short acting GLP-1 agonist may be administered less frequently than once per day to cover a time period relating to a single meal. In some embodiments, the short acting GLP-1 agonist may be administered as needed by a subject desiring to lose and/or to maintain weight.

The short acting GLP-1 agonist may be administered to a subject for any period of time, such as for a period of more than 1 month, more than 3 months, more than 6 months, 1 year, or more than 1.5 years, or more than 2 years or for the life of that subject. It may also be administered intermittently, such as once a day for one week and then skipping a period of time (e.g., days or weeks or months) and resuming again.

The short acting GLP-1 agonist may help a subject maintain or lose weight without the side effects associated with long acting agonists.

The advantage of the short acting GLP-1, and in particular ROSE-010, is that the dosage and timing of administration can be adapted to the needs of the subject to allow more flexibility in choice of treatment and assistance in losing or maintaining weight. Thus, a subject starting on ROSE-010 may use it initially before each meal of the day and as weight is lost reduce such usage to twice a day and then ultimately to once a day before the main meal of that subject. Holidays can be taken from usage if desired such as once a day during a one week period or more frequently depending on the subject and the desire for weight loss or maintenance. This flexibility increases the chance that a subject not only loses weight but also maintains a lower weight compared to when the subject started use of any GLP-1 agonist for such purpose. The subject then is in control of their weight loss and empowered to determine their own destiny in such manner. The subject may also thereby regulate the incidence of side effects if any. Compared to the long acting GLP-1 agonists the subject is able to get away from the feeling of dependency on the agonist and the side effects.

For example, a subject with a BMI of about 30 may take ROSE-010 3 times a day until their BMI has reduced to 27, and then just 2 times a day until their BMI is 25 at which point they may decide that is their preferred weight and take ROSE-010 just once a day thereon. Other subjects may be more aggressive and decide that they wish to use the ROSE-010 3 times a day until they are at a BMI of 25 and then reduce to twice a day to a BMI of 23 and then use it once a day thereafter.

In some embodiments, the subject may be an obese or an overweight subject. For example, the subject may have a BMI no less than 27, or no less than 28 or no less than 29 or no less than 30, or no less than 31. In some embodiments, the subject mat have a BMI of no less than 27 and no more than 35. In some embodiments, the subject may be a female. In some embodiments, the subject may be a male.

In some embodiments, the short acting GLP-1, such as ROSE-010, may be used for reducing a weight of an obese or an overweight subject desiring to reduce weight.

In some embodiments, the short acting GLP-1, such as ROSE-010, may be used for reducing a food consumption in a subject desiring to reduce food consumption, such as an obese or an overweight subject. For example, in some embodiments, administering the short acting GLP-1, such as ROSE-010, may reduce a daily calorie consumption by the subject by at least 20% or at least 25% or from 25% to 40% or from 27 to 39%.

In some embodiments, administering the short acting GLP-1, such as ROSE-010, may result in no side effects associated with the short acting GLP-1, such as ROSE-010, e.g. vomiting and/or nausea, or result in a side effect associated with the short acting GLP-1, such as ROSE-010, e.g. vomiting and/or nausea, that lasts no longer than no longer than the subject is exposed to the short acting GLP-1, such as ROSE-010, i.e. no longer than 2.5 hours or no longer than 2 hours.

### Prior to Meal and Multiple Doses

In a preferred embodiment, administration of a short-acting GLP-1 agonist acts quickly and its effects diminish within a few hours, making it ideally suited for administration around the main meal of the day. Accordingly, in one aspect, the present disclosure relates to a method of treating a subject comprising administering a formulation comprising a short-acting GLP-1 agonist prior to a meal. In some aspects, a short-acting GLP-1 agonist is administered about 10 minutes, about 15, minutes, about 20 minutes, about 30, minutes, about 45 minutes, about 60 minutes, about 10-60 minutes, about 15-50 minutes, or about 15-45 minutes prior to a meal.

In another aspect, a short-acting GLP-1 agonist may be administered multiple times in a single day. For example, a composition of a short-acting GLP-1 agonist may be administered prior to each meal. It may be administered twice a day, three times a day, or four times a day.

### Identifying Subjects Who Discontinued Treatment

The most common side effects of taking GLP-1 agonists are nausea and vomiting, which may persist so long as the subject is exposed to the GLP-1 agonist. This is particularly true of longer acting GLP-1 agonists. These persistent and continuous gastric side effects may cause many to discontinue using the GLP-1 agonists. In some embodiments, the present disclosure is directed to a method of treating a subject for weight loss comprising identifying a subject that discontinued treatment with a GLP-1 agonist and administering a formulation comprising a short-acting GLP-1 agonist to the subject. The discontinued GLP-1 agonist may be selected from semaglutide, tirzepatide, or liraglutide. In another aspect of the disclosure, the discontinued GLP-1 agonist was taken for at least 6 months, at least 9 months, or at least a year.

### Reducing Long-Acting GLP-1 Agonist Dosage

In another aspect, the present disclosure is directed to a method of administering a composition comprising a short acting GLP-1 agonist to a subject taking a long-acting GLP-1. Administration of the ROSE-010 composition can allow one to reduce the dosage of or the frequency of administering the long-acting GLP-1 agonist. Reducing the dosage and/or frequency of the longer acting GLP-1 agonist may simultaneously reduce the side effects associated with drug. Administration of the short acting GLP-1 agonist and the longer acting GLP-1 agonist may be done concurrently, or they may be administered at different times. Moreover, the short acting GLP-1 agonist may be administered multiple times per dosing period compared to the longer-acting GLP-1 agonist applied in the same dosing period. As noted above, the long-acting GLP-1 agonist may be administered at a frequency less than normal for that agonist, such that a daily administration can be reduced to once every two days, or three days or four days for example. The ROSE-010 composition can be then administered on those days when the long acting agonist is having little effects, such as on day two or day three or day four or some or all such days, and at a time when the subject is just about to take the main meal of the day. Preferably, such ROSE-010 is taken within about 30 minutes of such main meal. However, those in the art will recognize that some benefit is still obtained even if the ROSE-010 is taken at the start of such meal, especially if the meal is expected to last over an hour (such as a three course meal where the appetizers are served 30 minutes before a main course—in which case the subject will eat less of that main course).

For other long acting agonists that are given once per week or once per month they can similarly be reduced to being given just once per two weeks, or three weeks, or four weeks, or five weeks, or six weeks, or seven weeks, and to once per two months or three months or four months. In such case the ROSE-010 can be given again during periods when the long acting agonist is not expected to be active either multiple times a day, or once a day or less frequently like every other day.

For example, a subject with a BMI of about 30 may take a once a day long acting agonist until their BMI has reduced to 27, and then take that long acting agonist every other day and ROSE-010 on the days off once, twice or three times a day until their BMI is 25 at which point they may decide that is their preferred weight and take ROSE-010 just once a day thereafter. Other subjects may be more aggressive and decide that they wish to use the long acting agonist until they are at a BMI of 25 and then reduce to every other day with ROSE-010 until they have a BMI of 23 and then just ROSE-010 once a day thereafter.

### Reducing Adverse Side Effects

In a further aspect, the present disclosure is directed to a method for reducing the adverse side effects of a long acting GLP-1 agonist. In accordance with this aspect, the administration of the short acting GLP-1 agonist allows one to reduce the dosage of and/or the frequency of the long-acting GLP-1 agonist, which can reduce the adverse side effects of the long-acting GLP-1 agonist. This aspect of the disclosure may in some embodiments include administering to a subject a composition comprising a short-acting GLP-1 agonist and reducing either or both of amount or frequency of the dose of the long acting GLP-1 agonist administered to said subject to reduce the adverse side effects of said long acting GLP-1 agonist.

### Weight Maintenance Target Level

Administration of the short-acting GLP-1 agonist may be used to maintain a subject's weight. The subject may have attained their weight or a target weight with or without the use of a GLP-1 drug, such as a long acting GLP-1 agonist. In these embodiments, methods of the disclosure may include identifying a subject desiring to or in need of maintaining a particular weight, and administering to the subject a composition comprising a short-acting GLP-1 agonist. In another aspect, the disclosure relates to a method for treating an overweight or obese subject comprising administering a long acting GLP-1 agonist until the subject loses weight to a target level, then reducing or discontinuing dosing of the long acting GLP-1 agonist, and administering to the subject a composition comprising a short-acting GLP-1 agonist. In yet another aspect, the disclosure relates to a method of maintaining weight in a subject comprising identifying a subject that had lost weight while taking a GLP-1 agonist and administering to that subject a composition comprising a short-acting GLP-1 agonist.

### SELECTED SPECIFIC EMBODIMENTS

In addition to the aspects and embodiments disclosed elsewhere herein, the following particular embodiments are specifically contemplated.
1. A method of treating a subject comprising administering to said subject a composition comprising a short-acting GLP-1 agonist prior to a meal, wherein said composition provides an amount of the short-acting GLP-1 agonist that is effective for less than one day.
2. A method comprising identifying a subject that discontinued treatment with a longer acting GLP-1 agonist and administering to said subject a composition comprising shorter acting GLP-1 agonist
3. A method comprising identifying a subject that had previously taken and subsequently discontinued the GLP-1 agonist treatment, and administering to said subject a composition comprising short-acting GLP-1 agonist.
4. A method for reducing the amount of a long acting GLP-1 agonist administered to a subject; said method comprising administering to said subject a composition comprising a shorter acting GLP-1 agonist, and reducing either or both of amount or frequency of the dose of the long acting GLP-1 agonist administered to said subject.
5. A method for reducing at least one adverse side effect of a longer acting GLP-1 agonist administered to a subject; said method comprising administering to said subject a composition comprising a short-acting GLP-1 agonist and reducing either or both of amount or frequency of the dose of the long acting GLP-1 agonist administered to said subject to reduce the at least one adverse side effect of said long acting GLP-1 agonist.
6. A method for treating an overweight or obese subject, said method comprising administering a long acting GLP-1 agonist until the subject loses weight to a target level; then reducing or discontinuing dosing of said long acting GLP-1 agonist and administering to said subject a composition comprising a short-acting GLP-1 agonist.
7. A method comprising identifying a subject desiring to (or in need of) maintaining a particular weight, and administering to said subject a composition comprising a short-acting GLP-1 agonist.
8. A method of maintaining weight in a subject, said method comprising identifying a subject that had lost weight while taking a longer acting GLP-1 agonist and administering to said subject a composition comprising a short-acting GLP-1 agonist.
9. A method comprising identifying a patient diagnosed with, or at risk of, a cognitive disease or disorder and administering to said subject a composition comprising a short-acting GLP-1 agonist.
10. A method comprising identifying a patient diagnosed with, or at risk of, a neurodegenerative disease or disorder and administering to said subject a composition comprising a short-acting GLP-1 agonist.
11. A method of any of the preceding embodiments, wherein the short-acting GLP-1 agonist is also fast-acting.
12. A method of any of preceding embodiments, wherein the short-acting GLP-1 agonist comprises SEQ ID NO: 1.
13. A method of treating a subject comprising administering to said subject a composition comprising ROSE-010 prior to a meal, wherein said composition provides an amount of the short-acting GLP-1 agonist that is effective for less than one day.
14. A method comprising identifying a subject that discontinued treatment with a longer acting GLP-1 agonist and administering to said subject a composition comprising ROSE-010.
15. A method comprising identifying a subject that had previously taken and subsequently discontinued the GLP-1 agonist treatment, and administering to said subject a composition comprising ROSE-010.
16. A method for reducing the amount of a long acting GLP-1 agonist administered to a subject, said method comprising administering to said subject a composition comprising ROSE-010 and reducing either or both of amount or frequency of the dose of the long acting GLP-1 agonist administered to said subject.
17. A method for reducing at least one adverse side effect of a longer acting GLP-1 agonist administered to a subject; said method comprising administering to said subject a composition comprising ROSE-010 and reducing either or both of amount or frequency of the dose of the long acting GLP-1 agonist administered to said subject to reduce the at least one adverse side effect of said long acting GLP-1 agonist.
18. A method for treating an overweight or obese subject, said method comprising administering a long acting GLP-1 agonist until the subject loses weight to a target level, then reducing or discontinuing dosing of said long acting GLP-1 agonist and administering to said subject a composition comprising ROSE-010.
19. A method comprising identifying a subject desiring to (or in need of) maintaining a particular weight, and administering to said subject a composition comprising a ROSE-010.
20. A method of maintaining weight in a subject, said method comprising identifying a subject that had lost weight while taking a longer acting GLP-1 agonist and administering to said subject a composition comprising ROSE-010.
21. A method comprising identifying a patient diagnosed with, or at risk of, a cognitive disease or disorder and administering to said subject a composition comprising ROSE-010.
22. A method comprising identifying a patient diagnosed with, or at risk of, a neurodegenerative disease or disorder and administering to said subject a composition comprising ROSE-010.
23. A method of any of 1-22, wherein the short acting GLP-1 agonist is ROSE-010, exenatide, or lixisenatide.
24. A method of any of 1-23, wherein the long acting GLP-1 agonist is semaglutide, dulaglutide, tirzepatide, or liraglutide.
25. A method of any of 1-24, wherein administration of the short acting GLP-1 agonist reduces nausea relative to a method wherein only a long acting GLP-1 agonist is administered.
26. A method comprising identifying a subject desiring to (or in need of) reducing from a particular weight, and administering to said subject a composition comprising a ROSE-010. That ROSE-010 is administered to the subject initially between 1-3 times a day prior to meals and as weight is lost over time the amount of administration is reduced such that weight loss still continues and ultimately is maintained. The method may preferably allow no administration of the ROSE-010 during a one week period for 1, 2, 3, 4 or even 5 days which may be separated or continuous. Thus, the method allows a subject to determine their own dosing regimen that provides for weight loss or maintenance with minimal or no side effects.
27. A method of treating a subject desiring to lose weight or maintain their weight, comprising administering to said subject a composition comprising ROSE-010 prior to a meal in an amount sufficient to suppress the appetite of said subject.
28. A method comprising identifying a subject that discontinued treatment with a longer acting GLP-1 agonist selected from the group consisting of semaglutide, dulaglutide, tirzepatide, and liraglutide and administering to said subject a composition comprising ROSE-010 in an amount sufficient to suppress the appetite of said subject.
29. A method comprising identifying a subject that had previously taken and subsequently discontinued treatment with a GLP-1 agonist selected from the group consisting of semaglutide, dulaglutide, tirzepatide, and liraglutide, and administering to said subject a composition comprising ROSE-010 in an amount sufficient to suppress the appetite of said subject.
30. A method for reducing the amount of a long acting GLP-1 agonist selected from the group consisting of semaglutide, dulaglutide, tirzepatide, and liraglutide administered to a subject; said method comprising administering to said subject a composition comprising ROSE-010 in an amount sufficient to suppress the appetite of said subject, and reducing either or both of amount or frequency of the dose of the long acting GLP-1 agonist administered to said subject.
31. A method for reducing at least one adverse side effect of a GLP-1 agonist selected from the group consisting of semaglutide, dulaglutide, tirzepatide, and liraglutide administered to a subject; said method comprising administering to said subject a composition comprising ROSE-010 in an amount sufficient to suppress the appetite of said subject, and reducing either or both of amount or frequency of the dose of the GLP-1 agonist administered to said subject to reduce the at least one adverse side effect of said long acting GLP-1 agonist.
32. A method for treating an overweight or obese subject, said method comprising administering a GLP-1 agonist selected from the group consisting of semaglutide, dulaglutide, tirzepatide, and liraglutide until the subject loses weight to a target level; then reducing or discontinuing dosing of said GLP-1 agonist and administering to said subject a composition comprising ROSE-010 in an amount sufficient to suppress the appetite of said subject.
33. A method comprising identifying a subject desiring to maintain a particular weight, and administering to said subject a composition comprising ROSE-010 in an amount sufficient to suppress the appetite of said subject.
34. A method of maintaining weight in a subject, said method comprising identifying a subject that had lost weight while taking a GLP-1 agonist selected from the group consisting of semaglutide, dulaglutide, tirzepatide, and liraglutide and administering to said subject a composition comprising ROSE-010 in an amount sufficient to suppress the appetite of said subject.
35. A method comprising identifying a patient diagnosed with, or at risk of, a cognitive disease or disorder and administering to said subject a composition comprising ROSE-010 in an amount sufficient to suppress the appetite of said subject.
36. A method comprising identifying a patient diagnosed with, or at risk of, a neurodegenerative disease or disorder and administering to said subject a composition comprising ROSE-010 in an amount sufficient to suppress the appetite of said subject.
37. A method of reducing a weight of an overweight or obese subject, comprising administering to said subject a composition comprising ROSE-010 prior to a meal in an amount sufficient to suppress the appetite of said subject.
38. A method of reducing food consumption in a subject desiring to reduce food consumption, comprising administering to said subject a composition comprising ROSE-010 prior to a meal in an amount sufficient to suppress the appetite of said subject.
39. The method of 38, wherein said administering performed for 7 days once a day results in at least 25% reduction of calorie consumption by the subject.
40. The method of any one 1-19, wherein a single event of said administering does not result in a side effect associated with ROSE-010 lasting longer than 2.5 hours.
41. The method of 40, wherein the side effect is selected from the group consisting of vomiting, nausea and a combination thereof.
42. The method of any one of 1-41, wherein said administering is performed once a day.
43. The method of any one of 1-41, wherein said administering is performed at a frequency less than once a day.
44. The method of any one of 1-41, wherein said administering is performed as needed.
45. The method of any of 1-44, wherein the amount sufficient to suppress the appetite of said subject is selected from the group of 75 micrograms, 100 micrograms, 125 micrograms, 150 micrograms, 175 micrograms, 200 micrograms, 225 micrograms and 250 micrograms.
46. The method of any one of 1-44, wherein the amount sufficient to suppress the appetite of said subject is from 75 micrograms to 175 micrograms.
47. The method of any one of 1-44, wherein the amount sufficient to suppress the appetite of said subject is from 100 micrograms to 150 micrograms.
48. The method of any one of 45-47, wherein the ROSE-010 is administered subcutaneously or nasally.
49. The method of 48, wherein the ROSE-010 is administered at a time prior to a meal selected from 1, 10, 20, 30 and 40 minutes prior to said meal.
50. The method of 49, wherein the ROSE-010 is administered prior to a meal selected from 1, 2 or 3 meals in any day.
51. The method of 50, wherein the ROSE-010 is administered to a subject in an amount that is reduced over time from first administration.
52. The method of 51, wherein the ROSE-010 is administered to a subject initially in an amount of 2-3 times a day for a first period of time, and thereafter at 1-2 times a day for a second period of time, and thereafter at 1 time a day or less.
53. The method of 52, wherein the first and second periods of time are selected from the group of 1, 2, 3, 4, 5 and 6 months.
54. The method of any one of 1-53, wherein the subject is a female subject.
55. The method of any one of 1-54, wherein the subject has BMI of no less than 27 and no greater than 35.

The following examples are provided to further illustrate aspects and embodiments of the disclosure. These examples are non-limiting and should not be construed as limiting any aspects and embodiments of the disclosure.

### EXAMPLES

### Example 1

Clinical studies on long acting GLP-1 agonists such as semaglutide and tirzepatide have shown significant benefits in promoting weight loss in people with obesity and overweight. However, real world experience suggests that long acting GLP-1 agonists formulated for daily or weekly injection have relatively low tolerability and that persistence rates at 1-2 years are 50% or less. Studies suggest side effects such as nausea and vomiting limit long term treatment persistence for many subjects.

ROSE-010 is a rapid acting short half-life GLP-1 agonist that is being developed for appetite control in weight management. Due to its short half-life, periods of nausea are reduced allowing subjects to target mealtimes without feeling significant side effects between meals. Such a strategy may allow an “as needed” approach to GLP-1 therapy as part of a weight management strategy.

The impact of ROSE-010 on appetite was assessed in a randomised, placebo-controlled trial on 12 subjects with overweight and obesity. The population included males and females and the mean weight and BMI were 82.1 (8) kg and 29.8 (1.2) kg/m², respectively. The study was a crossover design with 2 doses of ROSE-010 (100 μg and 150 μg) and placebo. Each subject received one s.c. injection in each of the three treatment periods 30 minutes before a standardised meal and with a minimum of 2 days between each treatment. The treatment sequence allocation was double-blinded, i.e. it was not disclosed to the subjects or the treating clinician. The primary endpoint was amount/weight of food eaten at the standardised meal. Subjects were monitored for 6.5 hr post treatment and secondary endpoints included assessments of nausea, hunger, desire to eat and satiety during this period.

Response was dose dependent and the 150 ug ROSE-010 treatment resulted in a reduction of 16.7% food (gram) compared to the placebo treatment. Nausea was higher following the ROSE-010 treatments than with the placebo treatment but was reported as mild and resolved within 4 hours.

The results suggest that ROSE-010 may have potential in appetite control at mealtimes in subjects with overweight and obesity as an aid to weight control in addition to nutrition advice and education. The reduced period of nausea compared to long acting GLP-1 agonists may aid adherence to long term treatment.

### Example 2

### Summary

### Design

40 female subjects, healthy, 18-65 years of age, BMI (Body Mass Index) ≥27 & ≤35
Blinded with three parallel groups taking placebo (n=10), low dose ROSE-010 (n=15) and a high dose ROSE-010 (n=15) once daily for 7 days
Treatment 30 minutes before standardized meal taken at clinic, meals
Monitored for 6.5 hours post treatment

### Primary Endpoint

Food intake at meal following treatment
Food weight (g) & kcal value

### Other Endpoints

Measurement of ROSE-010 in blood over treatment
Body weight over 7 days
GI side effects, nausea (0-100 scale)
Hunger, satiety, prospective consumption, desire to eat, food palatability (0-100 scale)

### Safety

Treatments were given by subcutaneous injection 30 minutes before a standardized lunch. The treatment time was t0, the clock start from which other times were derived. The lunches varied daily and were targeted to be around 700-800 kcals or so. There was a sandwich or salad and protein, chips, a cookie and a fruit cup. After lunch, whatever remained uneaten was weighed and kcals calculated. The subjects for 6.5 hours with questionnaires on hunger, satiety (how full are you), prospective consumption (how much do you think you could eat now) and desire to eat (how much to you long to eat). Nausea was also measured hourly. All questionnaires were on 0-100 mm scales where 0 is none and 100 is highest imaginable for each parameter.

### Results

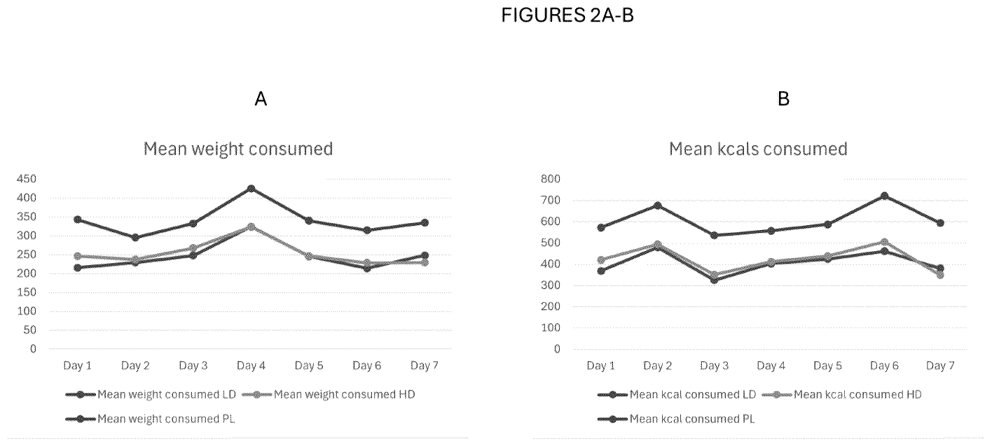

FIGS. 2A-B show that the ROSE-010 treated groups consistently eat less food weight and less calories than the placebo group. According to FIG. 2A, the ROSE-010 treated groups had daily lunch food weight consumption 22%-37% less than the placebo group. According to FIG. 2B, the ROSE-010 treated groups had a daily Lunch kcal consumption 27%-39% less than the placebo group.

Table 1 summarizes weight loss following 7 days treatment

TABLE 1

| Body Weight | 100 μg ROSE-010 | 150 μg ROSE-010 | Placebo |
|---|---|---|---|
| Baseline mean kg | 74.71 | 81.78 | 86.68 |
| Day 8 mean kg | 72.70 | 79.28 | 85.76 |
| Change Baseline | −2.01 | −2.50 | −0.92 |
| to Day 8 kg | | | |
| % Change Baseline | −2.68 | −3.03 | −1.07 |
| to Day 8 | | | |

Table 2 summarizes side effects associated with GLP-1s. According to Table 2, side effects associated with GLP-1s, nausea and vomiting, are mild and transient.

TABLE 2

| | | | Event | | |
|---|---|---|---|---|---|
| | Subjects | | Classification: | | |
| | with | Nausea | Mild, Moderate, | | Time to |
| | Nausea | Events | Severe | Vomiting | Resolve |
| 100 ug | 8 of 15 | 17 | Mild | No | 2.5 hours |
| Dose | | | | | |
| 150 ug | 6 of 15 | 21 | 4 mild; | 3 cases total; | 2.5 hours, |
| Dose | | | 2 moderate | 2 subjects | nausea |
| | | | | | 1 min, |
| | | | | | vomiting |

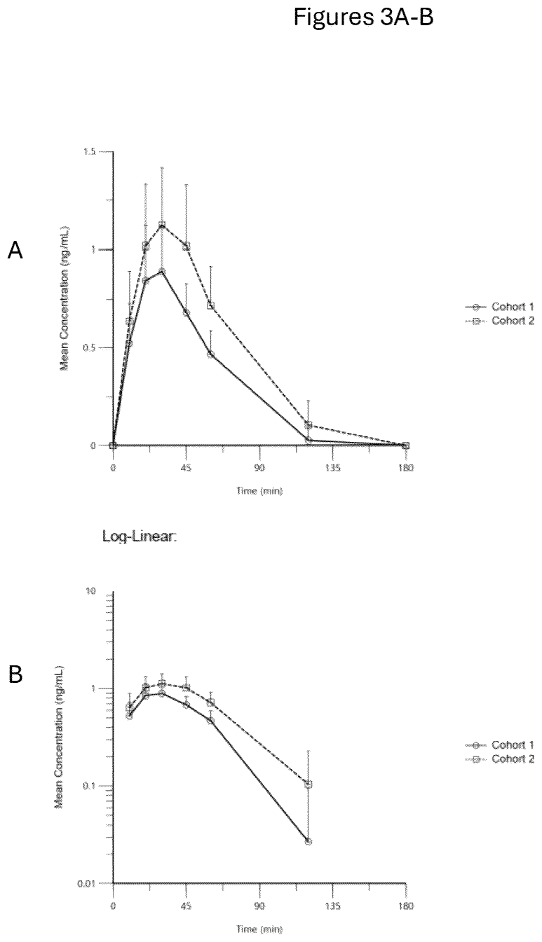

Nausea and vomiting only occur in about 50% of subjects. Where they do occur, they are dose dependent and resolve in 2.5 hours. In the lower 100 ug dose, GI side effects were all mild. Side effects associated with GLP-1s, nausea and vomiting, are limited to the period of drug exposure, see FIGS. 3A-B showing plasma concentrations of ROSE-010 over time following sub-cutaneous injection 100 ug (Cohort 1) and 150 ug (Cohort 2) ROSE-010.

### CONCLUSIONS

Weight loss does not require continuous exposure to GLP-1 receptor agonists with the associated continuous side effects of nausea and GI disturbances.

Cover with ROSE-010 for a 2-2.5 hour period at a single daily mealtime results in approximately 25% reduction in food consumption and associated energy intake at that meal.

The advantage of a 2-2.5 hour period is that:

Food consumption is substantially less at the target meal.

Side effects are only experienced during the drug exposure period.

The treatment flexibility allows a broad range of weight management options.

Treatment may be taken as needed, across a range. For example, at every daily meal for subjects wishing to lose weight to less than daily treatment at a single meal for those wishing to maintain weight.

The aspects, embodiments and examples described herein are illustrative and are not meant to be limitative. Variations of the foregoing embodiments, including alternatives, modifications and equivalents, are intended by the inventors to be encompassed by the present disclosure Citations listed in the present application are incorporated herein by reference in their entirety to the same extent as if individually incorporated by reference.

What is claimed is:

1. A method of treating a subject desiring to lose weight or maintain their weight, comprising administering to said subject a composition comprising a GLP-1 agonist having SEQ ID NO: 1 prior to a meal in an amount sufficient to suppress the appetite of said subject.

2. The method of claim 1, wherein the amount sufficient to suppress the appetite of said subject is selected from the group of 75 micrograms, 100 micrograms, 125 micrograms, 150 micrograms, 175 micrograms, 200 micrograms, 225 micrograms and 250 micrograms.

3. The method of claim 1, wherein the amount sufficient to suppress the appetite of said subject is from 75 micrograms to 175 micrograms.

4. The method of claim 1, wherein the GLP-1 agonist is administered subcutaneously or nasally.

5. The method of 1, wherein the GLP-1 agonist is administered at a time prior to a meal selected from 1, 10, 20, 30 and 40 minutes prior to said meal.

6. The method of claim 1, wherein the GLP-1 agonist is administered prior to a meal selected from 1, 2 or 3 meals in any day.

7. The method of claim 6, wherein the GLP-1 agonist is administered to the subject in an amount that is reduced over time from first administration.

8. The method of claim 7, wherein the GLP-1 agonist is administered to the subject initially in an amount of 2-3 times a day for a first period of time, and thereafter at 1-2 times a day for a second period of time, and thereafter at 1 time a day or less.

9. The method of claim 8, wherein the first and second periods of time are selected from the group of 1, 2, 3, 4, 5 and 6 months.

10. A method of reducing a weight of an overweight or obese subject, comprising administering to said subject a composition comprising a GLP-1 agonist having SEQ ID NO: 1 prior to a meal in an amount sufficient to suppress the appetite of said subject.

11. A method comprising identifying a subject desiring to maintain a particular weight, and administering to said subject a composition comprising a GLP-1 agonist having SEQ ID NO: 1 in an amount sufficient to suppress the appetite of said subject.

12. A method of reducing food consumption in a subject desiring to reduce food consumption, comprising administering to said subject a composition comprising a GLP-1 agonist having SEQ ID NO: 1 prior to a meal in an amount sufficient to suppress the appetite of said subject.

13. The method of claim 12, wherein said administering performed for 7 days once a day results in at least 25% reduction of calorie consumption by the subject.

14. The method of claim 12, wherein a single event of said administering does not result in a side effect associated with the GLP-1 agonist lasting longer than 2.5 hours.

15. The method of claim 14, wherein the side effect is selected from the group consisting of vomiting, nausea and a combination thereof.

16. The method of claim 12, wherein said administering is performed once a day.

17. The method of claim 12, wherein said administering is performed at a frequency less than once a day.

18. The method of claim 12, wherein said administering is performed as needed.

19. The method of claim 12, wherein the amount sufficient to suppress the appetite of said subject is selected from the group of 75 micrograms, 100 micrograms, 125 micrograms, 150 micrograms, 175 micrograms, 200 micrograms, 225 micrograms and 250 micrograms.

20. The method of claim 12, wherein the amount sufficient to suppress the appetite of said subject is from 75 micrograms to 175 micrograms.

21. The method of claim 12, wherein the amount sufficient to suppress the appetite of said subject is from 100 micrograms to 150 micrograms.

22. The method of claim 12, wherein the GLP-1 agonist is administered subcutaneously or nasally.

23. The method of claim 22, wherein the GLP-1 agonist is administered at a time prior to a meal selected from 1, 10, 20, 30 and 40 minutes prior to said meal.

24. The method of claim 23, wherein the GLP-1 agonist is administered prior to a meal selected from 1, 2 or 3 meals in any day.

25. The method of claim 24, wherein the GLP-1 agonist is administered to a subject in an amount that is reduced over time from first administration.

26. The method of claim 25, wherein the GLP-1 agonist is administered to a subject initially in an amount of 2-3 times a day for a first period of time, and thereafter at 1-2 times a day for a second period of time, and thereafter at 1 time a day or less.

27. The method of claim 26, wherein the first and second periods of time are selected from the group of 1, 2, 3, 4, 5 and 6 months.

28. The method of claim 12, wherein the subject is a female subject.

29. The method of claim 12, wherein the subject has BMI of no less than 27 and no greater than 35.

30. The method of claim 1, wherein a single event of said administering does not result in a side effect associated with the GLP-1 agonist lasting longer than 2.5 hours.


SEQUENCE LISTING (1 sequence)

SEQ ID NO: 1 - protein, 31 aa, synthetic construct
HVEGTFTSDVSSYLEGQAAKEFIAWLVKGRG